(19) United States
(12) Patent Application Publication
(10) Pub. No.: US 2026/0294537 A1
(43) Pub. Date: Oct. 1, 2026

(54) POWER SUPPLY FOR SURGICAL LASER CONSOLE

(71) Applicant: Lumenis Ltd., Yokneam IIit (IL)

(72) Inventors: Eitan Moshe Saba, Yokneam IIit, IL (US); Hila Best Weinreb, Nesher (IL)

(73) Assignee: Lumenis Ltd., Yokneam IIit (IL)

(21) Appl. No.: 19/634,242

(22) Filed: Mar. 31, 2026

Related U.S. Application Data
(60) Provisional application No. 63/781,044, filed on Mar. 31, 2025.

(51) Int. Cl. A61B 18/26; A61B 18/00

(52) U.S. Cl. CPC A61B 18/26; A61B 2018/00702; A61B 2018/00732; A61B 2018/00761; A61B 2018/00779

(57) ABSTRACT

The disclosure provides a surgical laser system including a surgical laser power supply and a surgical laser console. The surgical laser power supply can source higher electrical energy than it draws for a period of time in which the surgical laser console draws electrical energy from the surgical laser power supply to perform a surgical laser procedure, such as, laser lithotripsy or a laser enucleation of prostate (LEP) procedure.

### PRIORITY

This application claims the benefit of priority under 35 USC § 119 to United States Provisional Patent Application Serial No. 63/781,044, filed March 31, 2025, which is incorporated by reference herein in its entirety and for all purposes.

### TECHNICAL FIELD

The present disclosure generally relates to surgical laser systems. Particularly, but not exclusively, the present disclosure relates to surgical laser systems used in lithotripsy procedures and to power supplies for such laser systems.

### BACKGROUND

Medical lasers are used in a variety of procedures. Among a number of such procedures, laser energy is directed towards a target using an optical fiber as a conduit for the laser energy. One such procedure, to address renal calculi (e.g., kidney stones) is ureteral endoscopy, or lithotripsy. A typical lithotripsy procedure involves inserting an endoscopic (e.g., ureteroscope, or the like) into the urinary tract of a patient. A camera of the endoscope is used to locate the calculi for removal. AS part of the removal process, an optical fiber is inserted into the treatment area via a working channel of the endoscope. Laser energy is generated by a surgical laser console and the target is illuminated and/or irradiated with the laser energy via the optical fiber. The laser energy operates to disintegrate, dust, ablate, fragment and/or destroy the calculi.

Surgical laser consoles designed for laser lithotripsy procedures can utilize a number of lasing mediums to generate the laser energy. For example, holmium and thulium are common lasing mediums used to generate laser energy by such surgical laser consoles. Further, these surgical laser consoles are often configured to generate laser energy having tens (e.g., 30, 60, 90, or the like) or even hundreds (e.g., 120, 600, 500, or the like) Watts of optical power. Said differently, the surgical laser consoles can be configured to generate laser energy having pulse energy in the range of 0.5 Jules (J) to tens of Jules.

As will be appreciated by those of ordinary skill in the art, generating laser energy having such pulse energies or optical power requires a power supply configured to source multiple kilowatts of power. Thus, there is a need for advanced power supplies for surgical laser consoles.

### SUMMARY

This Summary is provided to introduce a selection of concepts in a simplified form that are further described below in the Detailed Description. This Summary is not intended to necessarily identify key features or essential features of the claimed subject matter, nor is it intended as an aid in determining the scope of the claimed subject matter.

The present disclosure provides a power supply for a surgical laser console configured to source sufficient power to the surgical laser console while sinking less power from a conventional electrical distribution system (e.g., electrical outlets, or the like). As outlined above, one use for surgical laser consoles is laser lithotripsy. Laser lithotripsy is often performed in hospital operating rooms and/or surgical centers, which can have varying power distribution capabilities. For example, many hospital operating rooms and/or surgical centers around the world do not have access to high-powered electrical distribution systems (e.g., configured to source electrical power in the range of multiple tens of Watts to thousands of Watts) necessary to power conventional surgical laser systems during a laser lithotripsy procedure. Accordingly, the present disclosure provides a power supply configured to source high enough power for conventional surgical laser systems to generate laser energy for a laser lithotripsy procedure while sinking power from electrical distribution systems not configured to source such high enough power.

The disclosure can be implemented as a surgical laser power supply. The surgical laser power supply can comprise a low-power supply configured to draw electrical energy from an electric power distribution network at a first power level; an energy storage array configured to draw electrical energy at the first power level and store electrical energy, wherein the energy storage array is further configured to release the stored electrical energy; a high-power outlet configured to receive a high-power plug of a surgical laser console; and a high-power supply, coupled to the energy storage array and the high-power outlet, and configured to source electrical energy at a second power level higher than the first power level.

In some embodiments of the surgical laser power supply, the first power level is less than or equal to 1,500 Watts and wherein the second power level is greater than 3,000 Watts and less than or equal to 8,000 Watts.

In some embodiments of the surgical laser power supply, the energy storage array comprises a plurality of electrochemical cells configured to store and release electrical energy.

In some embodiments of the surgical laser power supply, the electrochemical cells comprise Lithium-ion cells, Lithium-Iron Phosphate cells, Lithium-Manganese Oxide cells, Lithium-Cobalt Oxide cells, Lithium-Nickel-Manganese-Cobalt cells, or Lithium-Nickel-Cobalt-Aluminum Oxide cells.

In some embodiments, the surgical laser power supply can comprise a memory comprising instructions; and a processor coupled to the memory and configured to execute the instructions, which instructions when executed cause the surgical laser power supply to identify parameters of the surgical laser power supply, the parameters comprising at least a level of available energy of the energy storage array; receive, from a surgical laser console, an indication of parameters of the surgical laser console; and determine an available time with which the surgical laser power supply can source power to the surgical laser console based on the parameters of the surgical laser power supply and the parameters of the surgical laser console.

In some embodiments of the surgical laser power supply, the parameters of the surgical laser console comprise a pulse frequency, a pulse duration, and a pulse energy.

Some embodiments of the disclosure can be implemented as a surgical laser system. The surgical laser system can comprise a surgical laser power supply and a surgical laser console. The surgical laser power supply can comprise a low-power supply configured to draw electrical energy from an electric power distribution network at a first power level; an energy storage array configured to draw electrical energy at the first power level and store electrical energy, wherein the energy storage array is further configured to release the stored electrical energy; a high-power outlet; and a high-power supply, coupled to the energy storage array and the high-power outlet, and configured to source electrical energy at a second power level higher than the first power level. The surgical laser console can comprise a high-power plug configured to couple to the high-power outlet; a high-power supply configured to draw electrical energy from the surgical laser power source at the second power level via the high-power plug and the high-power outlet; a laser source configured to generate a pulsed laser beam; and optics configured to condition and couple the pulsed laser beam with an optical fiber for use in a surgical laser procedure.

In some embodiments of the surgical laser system, the first power level is less than or equal to 1,500 Watts and wherein the second power level is greater than 3,000 Watts and less than or equal to 8,000 Watts.

In some embodiments of the surgical laser system, the surgical laser power supply further comprises a memory comprising instructions; and a processor coupled to the memory and configured to execute the instructions, which instructions when executed cause the surgical laser power supply to identify parameters of the surgical laser power supply, the parameters comprising at least a level of available energy of the energy storage array; receive, from the surgical laser console, an indication of parameters of the surgical laser console; and determine an available time with which the surgical laser power supply can source power to the surgical laser console based on the parameters of the surgical laser power supply and the parameters of the surgical laser console.

In some embodiments of the surgical laser system, the parameters of the surgical laser console comprise a pulse frequency, a pulse duration, and a pulse energy.

In some embodiments of the surgical laser system, the instructions when executed further cause the surgical laser power supply to display the available time as part of a graphical user interface displayed on a display coupled to the surgical laser system.

In some embodiments of the surgical laser system, the surgical laser console further comprises console memory comprising console instructions; and a console processor coupled to the console memory and configured to execute the console instructions, which console instructions when executed cause the surgical laser power supply to receive, from an input device, indications of parameters or the surgical laser procedure; receive, from the surgical laser power supply, an indication of parameters of the surgical laser power supply, the parameters including at least a level of available energy of the electrical storage array; and determine an available lasing time based on the parameters of the surgical laser procedure and the parameters of the surgical laser power supply.

In some embodiments of the surgical laser system, the parameters of the surgical laser procedure comprise at least a pulsed laser beam power and the parameters of the surgical laser power supply comprise the level of available energy and the first level of power.

In some embodiments of the surgical laser system, the available lasing time is determined based on the following equation: T = 1/(E_{high} - E_{low})/E_{available}/60 where T is the available lasing time, E_{high} is pulsed laser beam power, E_{low} is the first level of power, and E_{available} is the level of available energy.

In some embodiments of the surgical laser system, the console instructions when executed further cause the surgical laser console to display the available lasing time as part of a console graphical user interface displayed on a console display coupled to the surgical laser system.

Some embodiments of the disclosure can be implemented as a method for a surgical laser system. The method can comprise receiving, from an input device, indications of parameters or a surgical laser procedure with which a surgical laser console is configured; receiving, from a surgical laser power supply, an indication of parameters of a surgical laser power supply coupled to the surgical laser procedure, the parameters including at least a level of available energy of an electrical storage array of the surgical laser power supply; and determining an available lasing time based on the parameters of the surgical laser procedure and the parameters of the surgical laser power supply.

In some embodiments of the method, the parameters of the surgical laser procedure comprise at least a pulsed laser beam power.

In some embodiments of the method, the surgical laser power supply comprises: a low-power supply configured to draw electrical energy from an electric power distribution network at a first power level, an energy storage array configured to draw electrical energy at the first power level and store electrical energy; and wherein the parameters of the surgical laser power supply comprise a level of available energy of the energy storage array and the first power level.

In some embodiments of the method, the available lasing time is determined based on the following equation: T = 1/(E_{high} - E_{low})/E_{available}/60 where T is the available lasing time, E_{high} is pulsed laser beam power, E_{low} is the first level of power, and E_{available} is the level of available energy.

In some embodiments, the method can comprise displaying the available time as part of a graphical user interface displayed on a display coupled to the surgical laser system.

### BRIEF DESCRIPTION OF THE FIGURES

To easily identify the discussion of any element or act, the most significant digit or digits in a reference number refer to the figure number in which that element is first introduced.

FIG. 1 illustrates a surgical laser system comprising a surgical laser console and a surgical laser power supply in accordance with embodiments of the disclosure.

FIG. 2 illustrates an example surgical laser console in accordance with embodiments of the disclosure.

FIG. 3 illustrates an example surgical laser power supply in accordance with embodiments of the disclosure.

FIG. 4 illustrates a method for a surgical laser console in accordance with embodiments of the disclosure.

FIG. 5 illustrates a method for a surgical laser power supply in accordance with embodiments of the disclosure.

FIG. 6 illustrates a computing system in accordance with embodiments of the disclosure.

### DETAILED DESCRIPTION

The foregoing has broadly outlined the features and technical advantages of the present disclosure such that the following detailed description of the disclosure may be better understood. It is to be appreciated by those skilled in the art that the embodiments disclosed may be readily utilized as a basis for modifying or designing other structures for carrying out the same purposes of the present disclosure. The novel features of the disclosure, both as to its organization and method of operation, together with further objects and advantages will be better understood from the following description when considered in connection with the accompanying figures. It is to be expressly understood, however, that each of the figures is provided for the purpose of illustration and description only and is not intended as a definition of the limits of the present disclosure.

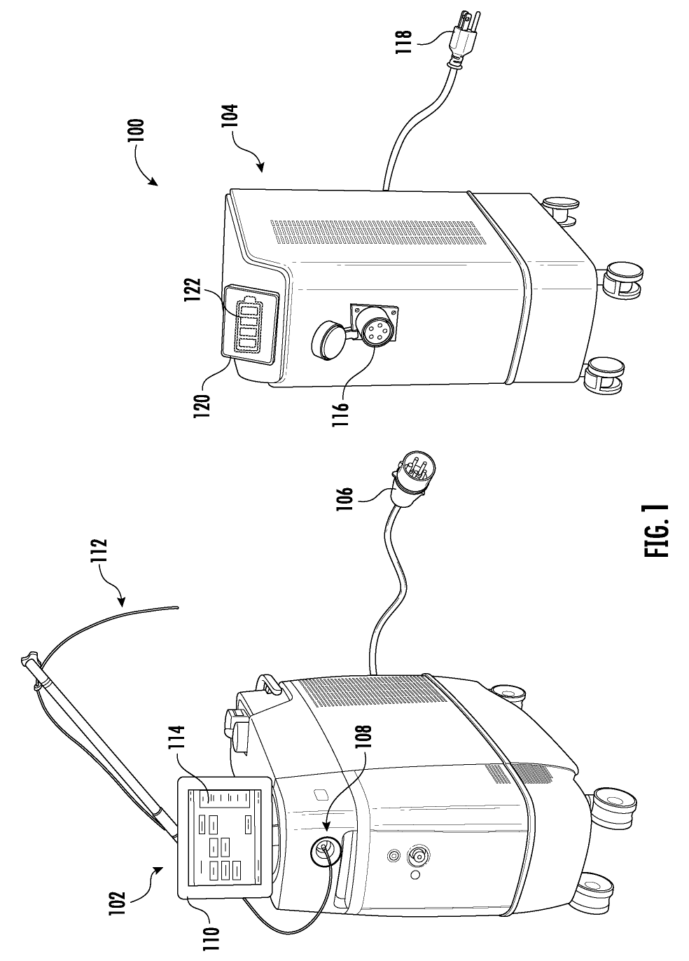

FIG. 1 illustrates an example surgical laser system 100, according to at least one embodiment of the disclosure. The surgical laser system 100 includes a surgical laser console 102 and a surgical laser power supply 104. In general, the surgical laser system 100 can be provided to perform surgical laser procedures in hospital operating rooms and/or surgical centers in which the electrical distribution system is insufficient to power the surgical laser console 102 during the procedure. For example, surgical laser system 100 can be provisioned to perform laser lithotripsy, laser enucleation of the prostate (LEP), or the like.

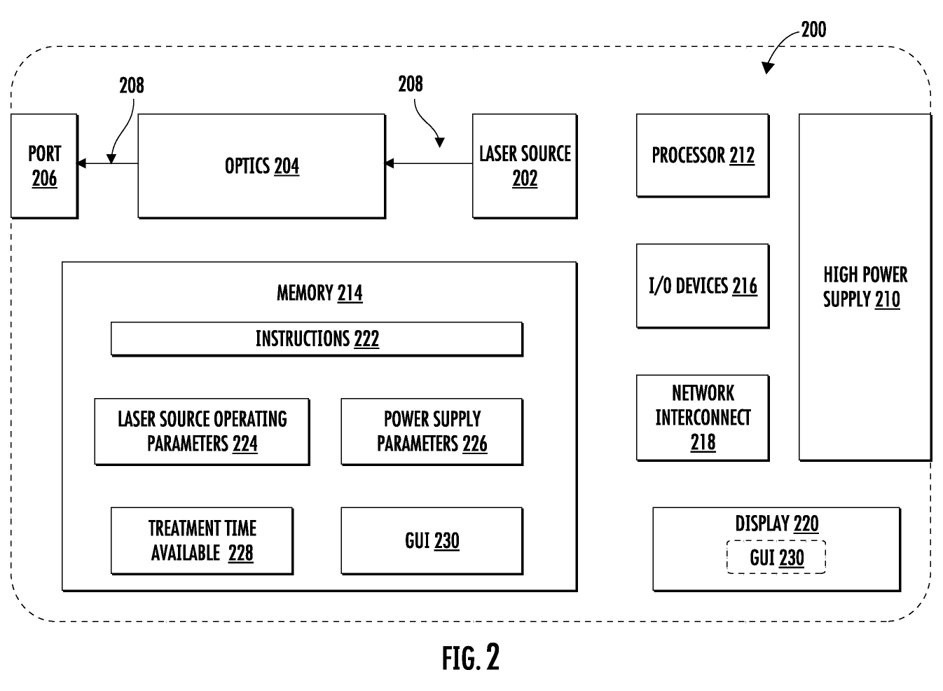

With some embodiments, the surgical laser console 102 can include a high-power plug 106, optical fiber port 108 and a display 110. Further, the surgical laser console 102 can include various laser sources and/or optical components (e.g., see FIG. 2) configured to generate laser energy and couple the laser energy to an optical fiber 112 via the optical fiber port 108. In general, the surgical laser console 102 requires electrical power in the range of multiple tens of Watts to thousands of Watts, or of hundreds of Watts to thousands of Watts. As a specific example, surgical laser console 102 may require (or draw) up to 8 kilowatts (kW) of electrical power during operation (e.g., when generating laser energy at maximum pulse energies). Further, during operation, a user can select via a graphical user interface (GUI) 114, the pulse energy to be generated during use. This selected pulse energy dictates the amount of electrical power with which the surgical laser console 102 will draw during use.

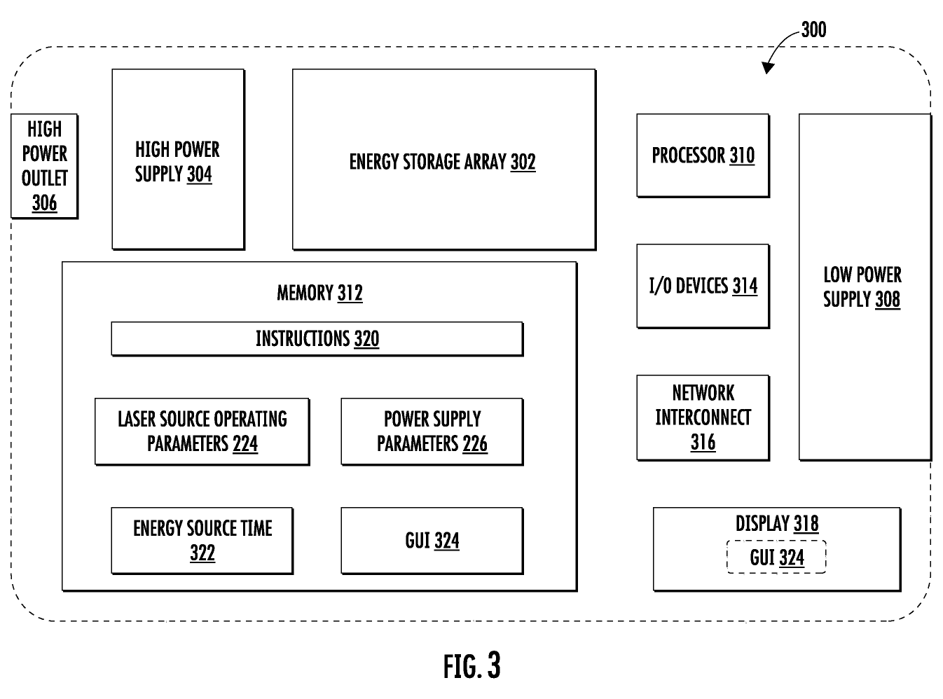

The surgical laser power supply 104 can include high-power outlet 116, low-power plug 118, and display 120. Further, the surgical laser power supply 104 can include various electrical components (e.g., see FIG. 3) configured to electrical power to supply to the surgical laser console 102 during a surgical laser procedure (e.g., laser lithotripsy, LEP, etc.) The high-power outlet 116 can be configured to receive the high-power plug 106 and to source electrical power at sufficient levels such that the surgical laser console 102 can generate laser energy having pulse power desired for the procedure. Further, the surgical laser power supply 104 can be coupled to a conventional electrical distribution system via the low-power plug 118. As outlined above, often conventional electrical power systems cannot source electrical power at levels with which the surgical laser console 102 may draw during use. Accordingly, the surgical laser power supply 104 is configured to source electrical power via the high-power outlet 116 and higher levels than it draws from the low-power plug 118. As such, the surgical laser system 100 can be provisioned to provide a surgical laser procedure in a hospital operating room and/or surgical suite with an electrical distributions system that cannot source high enough power for the procedure.

It is noted that the terms “high” and “low” as used herein do not have exact values associated with them but are instead used to identify relative levels. For example, the term “high-power” as used herein means higher power than the term “low-power.”

FIG. 2 illustrates an example surgical laser console 200, which can be provided according to examples of the present disclosure. In some embodiments, the surgical laser console 200 can be provided as the surgical laser console 102 of FIG. 1. With some embodiments, the surgical laser console 200 can be a commercially available surgical laser console, such as, for example, the Lumenis Pulse™ 120H Holmium Laser System available from Boston Scientific®.

The surgical laser console 200 includes a laser source 202, optics 204, and port 206. In general, the laser source 202 can comprise a lasing medium as well as pump lights (e.g., flash lamps, diodes, etc.) configured to excite the lasing medium causing the lasing medium to generate laser beam 208. The optics 204 can condition and/or focus the laser beam 208 on the port 206 where the laser beam 208 can be optically coupled to an optical fiber (e.g., optical fiber 112, the like) for use during a surgical laser procedure.

The surgical laser console 200 further includes high-power supply 210 configured to supply electrical power to components of the surgical laser console 200. Additionally, the surgical laser console 200 includes processor 212, memory 214, I/O input and/or output (I/O) devices 216, network interconnect 218, and display 220. The high-power supply 210 can be configured to supply power to the laser source 202 the laser source 202 such that the laser source 202 can generate the laser beam 208. Further, the high-power supply 210 can be configured to supply power to the other components (e.g., processor 212, etc.) of the surgical laser console 200.

As outlined above, the laser source 202 may draw up to several thousand Watts during use (e.g., 8 kW, or the like). In some embodiments, the high-power supply 210 can be configured to convert alternating current (AC) to direct current (DC) and further to regulate the DC voltage to a stable level for providing power to the laser source 202 and other components of the surgical laser console 200. In general, the high-power supply 210 can include any of a variety of circuitry and/or electronic component. For example, high-power supply 210 can include one or more transformer, rectifiers, filters, regulators, or the like.

The processor 212 may include circuity or processor logic, such as, for example, any of a variety of commercial processors. In some examples, processor 212 may include multiple processors, a multi-threaded processor, a multi-core processor (whether the multiple cores coexist on the same or separate dies), and/or a multi-processor architecture of some other variety by which multiple physically separate processors are in some way linked. Additionally, in some examples, the processor 212 may include graphics processing portions and may include dedicated memory, multiple-threaded processing and/or some other parallel processing capability. In some examples, the processor 212 may be an application specific integrated circuit (ASIC) or a field programmable integrated circuit (FPGA).

The memory 214 may include logic, a portion of which includes arrays of integrated circuits, forming non-volatile memory to persistently store data or a combination of non-volatile memory and volatile memory. It is to be appreciated, that the memory 214 may be based on any of a variety of technologies. In particular, the arrays of integrated circuits included in memory 214 may be arranged to form one or more types of memory, such as, for example, dynamic random-access memory (DRAM), NAND memory, NOR memory, or the like.

The I/O devices 216 can be any of a variety of devices to receive input and/or provide output. For example, I/O devices 216 can include, a keyboard, a mouse, a joystick, a foot pedal, a display, a touch enabled display, a haptic feedback device, an LED, or the like.

The network interconnect 218 can include logic and/or features to support a communication interface. For example, network interconnect 218 may include one or more interfaces that operate according to various communication protocols or standards to communicate over direct or network communication links. Direct communications may occur via use of communication protocols or standards described in one or more industry standards (including progenies and variants). For example, network interconnect 218 may facilitate communication over a bus, such as, for example, peripheral component interconnect express (PCIe), non-volatile memory express (NVMe), universal serial bus (USB), system management bus (SMBus), SAS (e.g., serial attached small computer system interface (SCSI)) interfaces, serial AT attachment (SATA) interfaces, or the like. Additionally, network interconnect 218 can include logic and/or features to enable communication over a variety of wired or wireless network standards. For example, network interconnect 218 may be arranged to support wired communication protocols or standards, such as, Ethernet, or the like. As another example, network interconnect 218 may be arranged to support wireless communication protocols or standards, such as, for example, Wi-Fi, Bluetooth, 5G, or the like.

The display 220 may include any of a variety of devices arranged to display graphical information, such as, a light emitting diode (LED) display, or the like). It is to be appreciated that although display 220 is depicted as incorporated into surgical laser console 200, the display 220 could be implemented separately from surgical laser console 200.

The memory 214 can include instructions 222, laser source operating parameters 224, power supply parameters 226, treatment time available 228, and GUI 230. During operation, processor 212 can execute instructions 222 to cause surgical laser console 200 to receive (e.g., via I/O devices 216, or the like) indications of operating parameters and store the operating parameters as laser source operating parameters 224. The laser source operating parameters 224 can include an indication of a procedure (e.g., lithotripsy, LEP, stone dusting, stone fragmenting, or the like), an indication of a laser energy (e.g., average power, pulse frequency, pulse energy, pulse duration, or the like).

Further, processor 212 can execute instructions 222 to cause laser source 202 to generate laser beam 208 based on the laser source operating parameters 224 and to draw power from a surgical laser power supply (e.g., surgical laser power supply 104, or the like) to operate the laser source 202.

Additionally, processor 212 can execute instructions 222 to receive (e.g., via network interconnect 218, or the like) indications of power supply parameters 226 from a surgical laser power supply (e.g., surgical laser power supply 104, or the like) and to send indications of the laser source operating parameters 224 to the surgical laser power supply. The power supply parameters 226 can include an indication of an electrical energy level of the surgical laser power supply, a maximum amount of electrical energy that can be drawn, an amount of electrical energy with which the surgical laser power supply draws from its power source, or the like.

Processor 212 can execute instructions 222 to derive treatment time available 228 based on laser source operating parameters 224 and power supply parameters 226. For example, processor 212 can execute instructions 222 to derive an amount of energy to be drawn from a power supply based on the procedure type and/or pulse parameters (e.g., frequency, duration, energy, etc.). Further, processor 212 can execute instructions 222 to derive a treatment time available 228 based on the amount of energy to be drawn and the energy level. As a specific example, processor 212 can execute instructions 222 to derive treatment time available 228 based on the following formula: T = 1/(E_{high} - E_{low})/E_{available}/60 where T is the lasing time available, E_{high} is the energy to be drawn from the power supply, E_{low} is the energy with which the power supply can draw from, and E_{available} is the energy available from the power supply.

Processor 212 can execute instructions 222 to generate GUI 230 comprising an indication of treatment time available 228. For example, the GUI 230 could include a visual indication of a number of minutes with which the surgical laser console 200 can continue lasing at current operating parameters.

FIG. 3 illustrates an example surgical laser power supply 300, which can be provided according to examples of the present disclosure. In some embodiments, the surgical laser power supply 300 can be provided as the surgical laser power supply 104 of FIG. 1.

The surgical laser power supply 300 includes an energy storage array 302, high-power supply 304, and high-power outlet 306.

In general, the energy storage array 302 can comprise electrochemical cells configured to store and release energy. For example, the energy storage array 302 can comprise Lithium-ion (Li-ion) cells, Lithium-Iron Phosphate (LiFePO4) cells, Lithium-Manganese Oxide (LMO) cells, Lithium-Cobalt Oxide (LCO) cells, Lithium-Nickel-Manganese-Cobalt (NMC) cells, or Lithium-Nickel-Cobalt-Aluminum Oxide (NCA) cells, or the like.

During operation, the energy storage array 302 can source DC electrical energy to the high-power supply 304. The high-power supply 304 can be configured to convert DC to AC and further to regulate the AC voltage to a stable level and provide the DC electrical energy at the high-power outlet 306. In general, the high-power supply 304 can include any of a variety of circuitry and/or electronic component. For example, high-power supply 304 can include one or more transistors, oscillators, transformers, filters, regulators, or the like.

With some embodiments, the energy storage array 302, the high-power supply 304, and the low-power supply 308 can cooperate to pass through electrical energy drawn from the electric power distribution network via the low-power supply 308 to the high-power supply 304.

As outlined above, a surgical laser console (e.g., the surgical laser console 102, surgical laser console 200, or the like) may draw up to several thousand Watts during use (e.g., 8 kW, or the like). In some embodiments, the high-power supply 304 can be configured to convert DC electrical energy stored in the energy storage array 302 to AC electrical energy at power levels sufficient to operate a surgical laser console.

In some embodiments, the energy storage array 302 can comprise sufficient capacity to operate a surgical laser console (e.g., surgical laser console 102, surgical laser console 200, etc.) for one or more procedures. For example, a typical laser lithotripsy procedure

The surgical laser power supply 300 further includes a low-power supply 308 configured to draw power from a low-power electrical distribution system. Additionally, the surgical laser power supply 300 includes processor 310, memory 312, I/O devices 314, network interconnect 316, and display 318. The low-power supply 308 can be configured to supply power to the energy storage array 302 such that the energy storage array 302 can store electrical energy. Further, the low-power supply 308 can be configured to supply power to the other components (e.g., processor 310, etc.) of the surgical laser power supply 300.

As noted, the low-power supply 308 is configured to draw electrical energy from a low-power supply electrical distribution system and to source electrical power to both the energy storage array 302 and other components of the surgical laser power supply 300. In some embodiments, the low-power supply 308 can be configured to convert alternating current (AC) to direct current (DC) and further to regulate the DC voltage to a stable level for providing power to the energy storage array 302 and other components of the surgical laser power supply 300. In general, the low-power supply 308 can include any of a variety of circuitry and/or electronic component. For example, low-power supply 308 can include one or more transformer, rectifiers, filters, regulators, or the like.

The processor 310 may include circuity or processor logic, such as, for example, any of a variety of commercial processors. In some examples, processor 310 may include multiple processors, a multi-threaded processor, a multi-core processor (whether the multiple cores coexist on the same or separate dies), and/or a multi-processor architecture of some other variety by which multiple physically separate processors are in some way linked. Additionally, in some examples, the processor 310 may include graphics processing portions and may include dedicated memory, multiple-threaded processing and/or some other parallel processing capability. In some examples, the processor 310 may be an application specific integrated circuit (ASIC) or a field programmable integrated circuit (FPGA).

The memory 312 may include logic, a portion of which includes arrays of integrated circuits, forming non-volatile memory to persistently store data or a combination of non-volatile memory and volatile memory. It is to be appreciated, that the memory 312 may be based on any of a variety of technologies. In particular, the arrays of integrated circuits included in memory 312 may be arranged to form one or more types of memory, such as, for example, dynamic random-access memory (DRAM), NAND memory, NOR memory, or the like.

The I/O devices 314 can be any of a variety of devices to receive input and/or provide output. For example, I/O devices 314 can include, a keyboard, a mouse, a joystick, a foot pedal, a display, a touch enabled display, a haptic feedback device, an LED, or the like.

The network interconnect 316 can include logic and/or features to support a communication interface. For example, network interconnect 316 may include one or more interfaces that operate according to various communication protocols or standards to communicate over direct or network communication links. Direct communications may occur via use of communication protocols or standards described in one or more industry standards (including progenies and variants). For example, network interconnect 316 may facilitate communication over a bus, such as, for example, peripheral component interconnect express (PCIe), non-volatile memory express (NVMe), universal serial bus (USB), system management bus (SMBus), SAS (e.g., serial attached small computer system interface (SCSI)) interfaces, serial AT attachment (SATA) interfaces, or the like. Additionally, network interconnect 316 can include logic and/or features to enable communication over a variety of wired or wireless network standards. For example, network interconnect 316 may be arranged to support wired communication protocols or standards, such as, Ethernet, or the like. As another example, network interconnect 316 may be arranged to support wireless communication protocols or standards, such as, for example, Wi-Fi, Bluetooth, 5G, or the like.

The display 318 may include any of a variety of devices arranged to display graphical information, such as, a light emitting diode (LED) display, or the like). It is to be appreciated that although display 318 is depicted as incorporated into surgical laser power supply 300, the display 318 could be implemented separately from surgical laser power supply 300.

The memory 312 can include instructions 320, laser source operating parameters 224, power supply parameters 226, energy source time 322, and GUI 324. During operation, processor 310 can execute instructions 320 to cause surgical laser power supply 300 to source electrical energy from the energy storage array 302 to a surgical laser console (e.g., surgical laser console 102, surgical laser console 200, or the like). Additionally, processor 310 can execute instructions 320 to determine power supply parameters 226. For example, processor 310 can execute instructions 320 to receive signals from energy storage array 302 comprising an indication of the electrical energy level of the energy storage array 302. Likewise, processor 310 can execute instructions 320 to determine an amount of electrical energy that can be drawn from the low-power supply 308.

Further, processor 310 can execute instructions 320 to receive (e.g., via network interconnect 316, or the like) indications of laser source operating parameters 224 from a surgical laser console (e.g., surgical laser console 102, surgical laser console 200, or the like) and to send power supply parameters 226 to the surgical laser console.

Processor 310 can execute instructions 320 to derive energy source time 322 based on laser source operating parameters 224 and power supply parameters 226 as outlined above. Further, processor 310 can execute instructions 320 to generate GUI 324 comprising an indication of energy source time 322. For example, the GUI 324 could include a visual indication of a number of minutes with which the surgical laser power supply 300 can continue sourcing power to a connected surgical laser console at current operating parameters of the surgical laser console.

FIG. 4 illustrates an example method 400, according to at least one embodiment of the disclosure. In some examples, the method 400 can be implemented by a surgical laser console, such as the surgical laser console 200 of FIG. 2 or the surgical laser console 102 of FIG. 1. The method 400 is described with reference to the surgical laser console 200 of FIG. 2 and the surgical laser power supply 300 of FIG. 3. However, it is to be appreciated that the method 400 could be implemented by another surgical laser system different than one comprised of surgical laser console 200 and surgical laser power supply 300.

The method 400 can begin at block 402. At block 402 “receive, from an I/O device, indications of parameters of a surgical laser procedure” parameters of a surgical laser procedure can be received from an I/O device of a surgical laser console. For example, a user of the surgical laser console 200 can input parameters for a surgical laser procedure (e.g., lithotripsy, LEP, etc.) using I/O devices 216. Likewise, processor 212 can execute instructions 222 to receive indications of the parameters from the I/O devices 216 and store the parameters as laser source operating parameters 224.

Continuing to block 404 “receive, from a surgical laser power supply, an indication of parameters of the surgical laser power supply, the parameters including at least a level of available energy” an indication of parameters, including a level of available electrical energy, can be received from a surgical laser power supply. For example, processor 212 can execute instructions 222 to receive, from a surgical laser power supply (e.g., surgical laser power supply 104, surgical laser power supply 300, or the like), indications of parameters of the surgical laser power supply and store the parameters as power supply parameters 226. For example, processor 212 can execute instructions 222 to receive indications of a level of available electrical energy in the energy storage array 302 and the amount of electrical energy with which the low-power supplies 308 can draw.

Continuing to block 406 “determine an available lasing time based on the parameters” a lasing time based on the console parameters and the power supply parameters can be determined. For example, processor 212 can execute instructions 222 to determine a time with which the laser source 202 can generate laser beam 208 based on laser source operating parameters 224 and power supply parameters 226 and store the time as treatment time available 228.

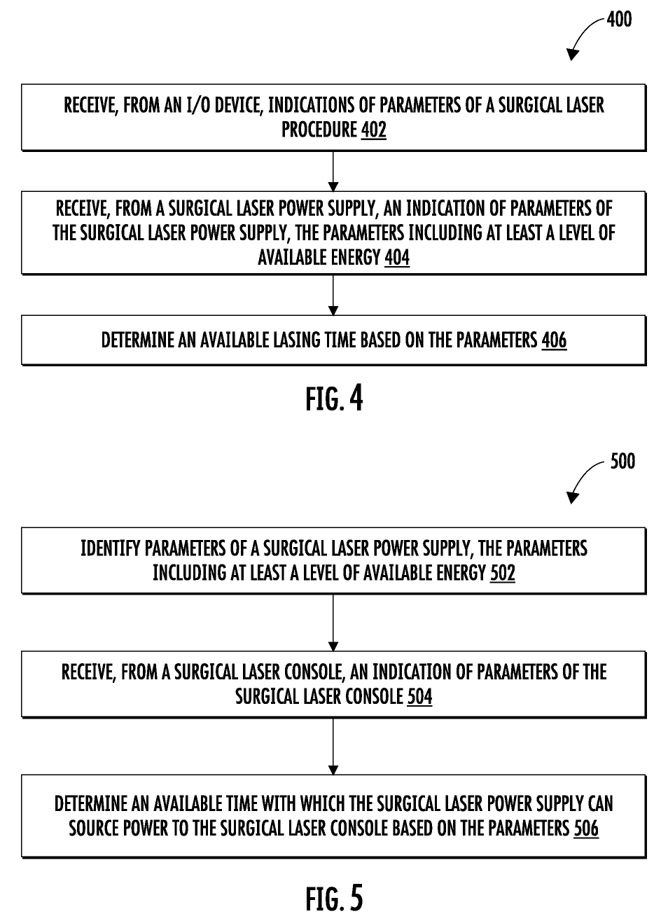

FIG. 5 illustrates an example method 500, according to at least one embodiment of the disclosure. In some examples, the method 500 can be implemented by a surgical laser power supply, such as the surgical laser power supply 300 of FIG. 3 or the surgical laser power supply 104 of FIG. 1. The method 500 is described with reference to the surgical laser console 200 of FIG. 2 and the surgical laser power supply 300 of FIG. 3. However, it is to be appreciated that the method 500 could be implemented by another surgical laser system different than one comprised of surgical laser console 200 and surgical laser power supply 300.

The method 500 can begin at block 502. At block 502 “identify parameters of a surgical laser power supply, the parameters including at least a level of available energy” parameters of a surgical laser power supply can be identified. For example, processor 310 can execute instructions 320 to identify a level of available electrical energy from the energy storage array 302, an amount of electrical energy that the low-power supply 308 can draw, and/or an amount of electrical energy with which the high-power supply 304 can source and store the parameters as power supply parameters 226.

Continuing to block 504 “receive, from a surgical laser console, an indication of parameters of the surgical laser console” an indication of parameters of a surgical laser console can be received from the surgical laser console. For example, processor 310 can execute instructions 320 to receive, from a surgical laser console (e.g., surgical laser console 102, surgical laser console 200, or the like), indications of laser source operating parameters 224.

Continuing to block 506 “determine an available time with which the surgical laser power supply can source power to the surgical laser console based on the parameters” a time with which the surgical laser power source can source power can be determined based on the console parameters and the power supply parameters. For example, processor 310 can execute instructions 320 to determine a time with which the surgical laser power supply 300 can source electrical energy to the surgical laser console 200 based on laser source operating parameters 224 and power supply parameters 226 and store the time as energy source time 322.

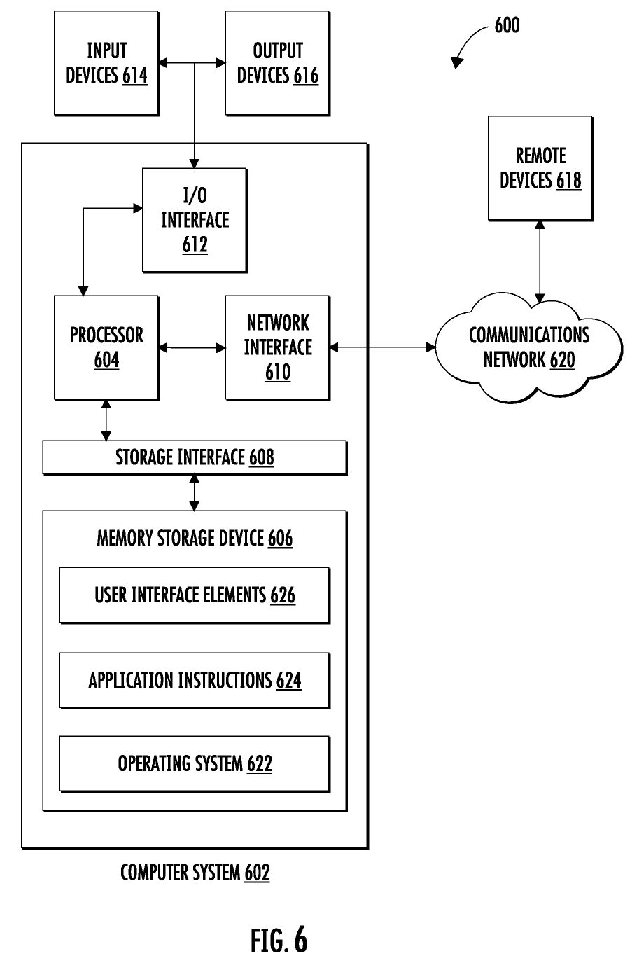

FIG. 6 is a block diagram of a computing environment 600 including a computer system 602 for implementing embodiments consistent with the present disclosure. In some embodiments, the computing environment 600, or portion thereof (e.g., the computer system 602) may comprise or be comprised in a surgical laser console (e.g., surgical laser console 102, surgical laser console 200, or the like) or a surgical laser power supply (e.g., surgical laser power supply 104, surgical laser power supply 300, or the like). Accordingly, in various embodiments, computer system 602 may determine lasing time and/or energy source time as outlined herein.

The computer system 602 may include a central processing unit (“CPU” or “processor”) 604. The processor 604 may include at least one data processor for executing instructions and/or program components for executing user or system-generated processes. A user may include a person, a person using a device such as those included in this disclosure, or another device. The processor 604 may include specialized processing units such as integrated system (bus) controllers, memory management control units, floating point units, graphics processing units, neural processing units, digital signal processing units, etc. The processor 604 may be disposed in communication with input devices 614 and output devices 616 via I/O interface 612. The I/O interface 612 may employ communication protocols/methods such as, without limitation, audio, analog, digital, stereo, IEEE-1394, serial bus, Universal Serial Bus (USB), infrared, PS/2, BNC, coaxial, component, composite, Digital Visual Interface (DVI), high-definition multimedia interface (HDMI), Radio Frequency (RF) antennas, S-Video, Video Graphics Array (VGA), IEEE 802.n /b/g/n/x, Bluetooth, cellular (e.g., Code-Division Multiple Access (CDMA), High-Speed Packet Access (HSPA+), Global System For Mobile Communications (GSM), Long-Term Evolution (LTE), or the like), etc.

Using the I/O interface 612, computer system 602 may communicate with input devices 614 and output devices 616. In some embodiments, the processor 604 may be disposed in communication with a communications network 620 via a network interface 610. In various embodiments, the communications network 620 may be utilized to communicate with a remote memory storage device 606, such as for accessing look-up tables, performing updates, or utilizing external resources. The network interface 610 may communicate with the communications network 620. The network interface 610 may employ connection protocols including, without limitation, direct connect, Ethernet (e.g., twisted pair 10/100/1000 Base T), Transmission Control Protocol/Internet Protocol (TCP/IP), token ring, IEEE 802.11a/b/g/n/x, etc.

The communications network 620 can be implemented as one of the different types of networks, such as intranet or Local Area Network (LAN), Closed Area Network (CAN) and such. The communications network 826 may either be a dedicated network or a shared network, which represents an association of the different types of networks that use a variety of protocols, for example, Hypertext Transfer Protocol (HTTP), CAN Protocol, Transmission Control Protocol/Internet Protocol (TCP/IP), Wireless Application Protocol (WAP), etc., to communicate with each other. Further, the communications network 620 may include a variety of network devices, including routers, bridges, servers, computing devices, storage devices, etcetera. In some embodiments, the processor 604 may be disposed in communication with a memory storage device 606 via a storage interface 608. The storage interface 608 may connect to memory storage device 606 including, without limitation, memory drives, removable disc drives, etc., employing connection protocols such as Serial Advanced Technology Attachment (SATA), Integrated Drive Electronics (IDE), IEEE-1394, Universal Serial Bus (USB), fiber channel, Small Computer Systems Interface (SCSI), etc. The memory drives may further include a drum, magnetic disc drive, magneto-optical drive, optical drive, Redundant Array of Independent Discs (RAID), solid-state memory devices, solid-state drives, etcetera.

Furthermore, memory storage device 606 may include one or more computer-readable storage media utilized in implementing embodiments consistent with the present disclosure. Generally, a computer-readable storage medium refers to any type of physical memory on which information or data readable by a processor may be stored. Thus, a computer-readable storage medium may store instructions for execution by one or more processors, including instructions for causing the processor(s) to perform steps or stages consistent with the embodiments described herein. The term “computer-readable medium” should be understood to include tangible items and exclude carrier waves and transient signals, i.e., non-transitory. Examples include Random Access Memory (RAM), Read-Only Memory (ROM), volatile memory, non-volatile memory, hard drives, Compact Disc (CD) ROMs, Digital Video Disc (DVDs), flash drives, disks, and any other known physical storage media.

The memory storage device 606 may store a collection of program or database components, including, without limitation, an operating system 622, application instructions 624, and user interface elements 626. In various embodiments, the operating system 622 may facilitate resource management and operation of the computer system 602. Examples of operating systems include, without limitation, APPLE^{®} MACINTOSH^{®} OS X^{®}, UNIX^{®}, UNIX-like system distributions (E.G., BERKELEY SOFTWARE DISTRIBUTION^{®} (BSD), FreeBSD^{®}, NetBSD^{®}, OpenBSD^{®}, etc.), LINUX^{®} DISTRIBUTIONS (E.G., RED HAT^{®}, UBUNTU^{®}, KUBUNTU^{®}, etc.), IBM^{®}OS/2^{®}, MICROSOFT^{®} WINDOWS^{®} (XP^{®}, VISTA^{®}/7/8, 10 etc.), APPLE^{®} IOS^{®}, GOOGLE^{TM} ANDROID^{TM}, BLACKBERRY^{®} OS, or the like.

The application instructions 624 may include instructions that when executed by the processor 604 cause the processor 604 to perform one or more techniques, steps, procedures, and/or methods described herein, such to determine lasing time and/or energy source time (e.g., treatment time available 228, energy source time 322, or the like). For example, application instructions 624, when executed by processor 604 can cause processor 604 to perform the method 400 and/or the method 500.

The user interface elements 626 may facilitate display, execution, interaction, manipulation, or operation of program components through textual or graphical facilities. For example, user interfaces may provide computer interaction interface elements on a display system operatively connected to the computer system 602, such as cursors, icons, checkboxes, menus, scrollers, windows, widgets, etcetera. The user interface elements 626 may be employed by application instructions 624 and/or operating system 622 to provide, for example, a user interface with which a user can interact with computer system 602. In some embodiments, the user interface elements 626 may be integrated with the display (e.g., display 110, display 120, or the like).

Terms used herein should be accorded their ordinary meaning in the relevant arts, or the meaning indicated by their use in context, but if an express definition is provided, that meaning controls.

Herein, references to "one embodiment" or "an embodiment" do not necessarily refer to the same embodiment, although they may. Unless the context clearly requires otherwise, throughout the description and the claims, the words "comprise," "comprising," and the like are to be construed in an inclusive sense as opposed to an exclusive or exhaustive sense; that is to say, in the sense of "including, but not limited to." Words using the singular or plural number also include the plural or singular number respectively, unless expressly limited to a single one or multiple ones. Additionally, the words "herein," "above," "below" and words of similar import, when used in this application, refer to this application as a whole and not to any particular portions of this application. When the claims use the word "or" in reference to a list of two or more items, that word covers all of the following interpretations of the word: any of the items in the list, all of the items in the list and any combination of the items in the list, unless expressly limited to one or the other. Any terms not expressly defined herein have their conventional meaning as commonly understood by those having skill in the relevant art(s).

What is claimed is:

1. A surgical laser power supply, comprising:
a low-power supply configured to draw electrical energy from an electric power distribution network at a first power level;
an energy storage array configured to draw electrical energy at the first power level and store electrical energy, wherein the energy storage array is further configured to release the stored electrical energy;
a high-power outlet configured to receive a high-power plug of a surgical laser console; and
a high-power supply, coupled to the energy storage array and the high-power outlet, and configured to source electrical energy at a second power level higher than the first power level.

2. The surgical laser power supply of claim 1, wherein the first power level is less than or equal to 1,500 Watts and wherein the second power level is greater than or equal to 3,000 Watts and less than or equal to 8,000 Watts.

3. The surgical laser power supply of claim 1, wherein the energy storage array comprises a plurality of electrochemical cells configured to store and release electrical energy.

4. The surgical laser power supply of claim 3, wherein the electrochemical cells comprise
Lithium-ion cells, Lithium-Iron Phosphate cells, Lithium-Manganese Oxide cells, Lithium-Cobalt Oxide cells, Lithium-Nickel-Manganese-Cobalt cells, or Lithium-Nickel-Cobalt-Aluminum Oxide cells.

5. The surgical laser power supply of claim 1, further comprising:
a memory comprising instructions; and
a processor coupled to the memory and configured to execute the instructions, which instructions when executed cause the surgical laser power supply to:
identify parameters of the surgical laser power supply, the parameters comprising at least a level of available energy of the energy storage array;
receive, from a surgical laser console, an indication of parameters of the surgical laser console; and
determine an available time with which the surgical laser power supply can source power to the surgical laser console based on the parameters of the surgical laser power supply and the parameters of the surgical laser console.

6. The surgical laser power supply of claim 5, wherein the parameters of the surgical laser console comprise a pulse frequency, a pulse duration, and a pulse energy.

7. A surgical laser system comprising:
a surgical laser power supply, comprising:
a low-power supply configured to draw electrical energy from an electric power distribution network at a first power level;
an energy storage array configured to draw electrical energy at the first power level and store electrical energy, wherein the energy storage array is further configured to release the stored electrical energy;
a high-power outlet; and
a high-power supply, coupled to the energy storage array and the high-power outlet, and configured to source electrical energy at a second power level higher than the first power level; and
a surgical laser console, comprising:
a high-power plug configured to couple to the high-power outlet;
a high-power supply configured to draw electrical energy from the surgical laser power source at the second power level via the high-power plug and the high-power outlet;
a laser source configured to generate a pulsed laser beam; and
optics configured to condition and couple the pulsed laser beam with an optical fiber for use in a surgical laser procedure.

8. The surgical laser system of claim 7, wherein the first power level is less than or equal to 1,500 Watts and wherein the second power level is greater than or equal to 3,000 Watts and less than or equal to 8,000 Watts.

9. The surgical laser system of claim 7, wherein the surgical laser power supply further comprises:
a memory comprising instructions; and
a processor coupled to the memory and configured to execute the instructions, which instructions when executed cause the surgical laser power supply to:
identify parameters of the surgical laser power supply, the parameters comprising at least a level of available energy of the energy storage array;
receive, from the surgical laser console, an indication of parameters of the surgical laser console; and
determine an available time with which the surgical laser power supply can source power to the surgical laser console based on the parameters of the surgical laser power supply and the parameters of the surgical laser console.

10. The surgical laser system of claim 9, wherein the parameters of the surgical laser console comprise a pulse frequency, a pulse duration, and a pulse energy.

11. The surgical laser system of claim 10, wherein the instructions when executed further cause the surgical laser power supply to display the available time as part of a graphical user interface displayed on a display coupled to the surgical laser system.

12. The surgical laser system of claim 7, wherein the surgical laser console further comprises:
console memory comprising console instructions; and
a console processor coupled to the console memory and configured to execute the console instructions, which console instructions when executed cause the surgical laser power supply to:
receive, from an input device, indications of parameters or the surgical laser procedure;
receive, from the surgical laser power supply, an indication of parameters of the surgical laser power supply, the parameters including at least a level of available energy of the electrical storage array; and
determine an available lasing time based on the parameters of the surgical laser procedure and the parameters of the surgical laser power supply.

13. The surgical laser system of claim 12, wherein the parameters of the surgical laser procedure comprise at least a pulsed laser beam power and the parameters of the surgical laser power supply comprise the level of available energy and the first level of power.

14. The surgical laser system of claim 13, wherein the available lasing time is determined based on the following equation: T = 1/(E_{high} - E_{low})/E_{available}/60 where T is the available lasing time, E_{high} is pulsed laser beam power, E_{low} is the first level of power, and E_{available} is the level of available energy.

15. The surgical laser system of claim 12, wherein the console instructions when executed further cause the surgical laser console to display the available lasing time as part of a console graphical user interface displayed on a console display coupled to the surgical laser system.

16. A method for a surgical laser system, comprising:
receiving, from an input device, indications of parameters or a surgical laser procedure with which a surgical laser console is configured;
receiving, from a surgical laser power supply, an indication of parameters of a surgical laser power supply coupled to the surgical laser procedure, the parameters including at least a level of available energy of an electrical storage array of the surgical laser power supply; and
determining an available lasing time based on the parameters of the surgical laser procedure and the parameters of the surgical laser power supply.

17. The method of claim 16, wherein the parameters of the surgical laser procedure comprise at least a pulsed laser beam power.

18. The method of claim 16, wherein the surgical laser power supply comprises: a low-power supply configured to draw electrical energy from an electric power distribution network at a first power level, an energy storage array configured to draw electrical energy at the first power level and store electrical energy; and wherein the parameters of the surgical laser power supply comprise a level of available energy of the energy storage array and the first power level.

19. The method of claim 18, wherein the available lasing time is determined based on the following equation: T = 1/(E_{high} - E_{low})/E_{available}/60 where T is the available lasing time, E_{high} is pulsed laser beam power, E_{low} is the first level of power, and E_{available} is the level of available energy.

20. The method of claim 16, further comprising displaying the available time as part of a graphical user interface displayed on a display coupled to the surgical laser system.
